(19) United States
(12) Patent Application Publication
(10) Pub. No.: US 2003/0098362 A1
(43) Pub. Date: May 29, 2003

(54) Portable diffusing perfume bottle

(76) Inventor: Tang-Jung Chuang, Taipei City (TW)

Correspondence Address: Tang-Jung Chuang, PO BOX 7-820, TAIPEI, TW

(21) Appl. No.: 09/960,138

(22) Filed: Sep. 24, 2001

(51) Int. Cl. A61L 9/04

(52) U.S. Cl. 239/44

(57) ABSTRACT

The invention relates to a portable perfume-diffusing bottle, consisting of a cap, a plug, a core pillar, and a bottle, characterized in that a partitioned inwardly depressed serrated ring is situated at a central opening of the plug, and the inwardly depressed teeth has a vertical ventilation passage so that the core pillar passes through the central opening of the plug, and is engaged in position. By means of the crevice passages on the transversal surface of the inner teeth, the perfume in the bottle will be directed to the outside airflow, and the core pillar will absorb the scented liquid and release it to the outside airflow which causes the perfume diffusion to start as it passes smoothly into the outside air and circulates. Such arrangement accomplishes the features of the practical application of circulating the diffused perfume into the air by circulation.

### BACKGROUND OF THE INVENTION

It is human nature that people desire to be beautiful, and it would not be an exaggeration to say that perfume speaks for the e-generation nowadays. At present, when perfume is worn, the user generally moisten or gently rub a tiny amount of liquid inside the perfume bottle onto the user's skin so that the diffusion of the perfume expresses the user's personality. Knowing that famous brands are continually pursuing a fashioned taste to make profit from the vast consumer's market, and completely neglecting the rights of consumers with allergic conditions, as well as younger or older users whose skin cannot adapt or may even cause skin diseases by the irritation of the perfume, therefore the use of cosmetic perfume keeps this minority of users from being able to enjoy their equal love for cosmetics and fragrance.

In light of the inconvenience mentioned above, the inventor of the invention broke through the traditional technology and shortcomings, thus invented the invention for the benefit of the neglected minority of users.

### SUMMARY OF THE INVENTION

The main object of this invention is to provide a portable diffusing perfume bottle through the operation of the components including a cap, a plug, a core pillar, and a bottle; it has the feature of perfume diffusion by circulation of the air to attain the practical effectiveness.

The secondary objective of this invention is to provide a portable diffusing perfume bottle, more particularly the perfume bottle comprising a central opening with partitioned inwardly-depressed inner serrated ring, and said inwardly depressed teeth has a vertical ventilation passage such that the core pillar passes through the central opening of the plug and is engaged in position.

Another objective of this invention is to provide a portable diffusing perfume bottle, wherein the crevice passages on the transversal surface of the inner teeth, the perfume in the bottle will be directed to the outside airflow, and the core pillar will absorb the scented liquid and release it to the outside airflow which brings the fragrant function into play as it passes smoothly into the outside air and circulates. Such arrangement accomplishes the features of the practical application of circulating the diffused perfume into the air by circulation.

### BRIEF DESCRIPTION OF THE DRAWINGS

FIG. 1 is shows the assembled structure of the invention;

FIG. 2 is shows the assembled parts of the structure according to the invention;

FIG. 3 is shows the cross-section diagram of the structure of the invention,

FIG. 4 is shows the cross-sectional diagram a preferred embodiment of the invention.

### DELTAILED DESCRIPTION OF THE PREFERRED EMBODIMENT

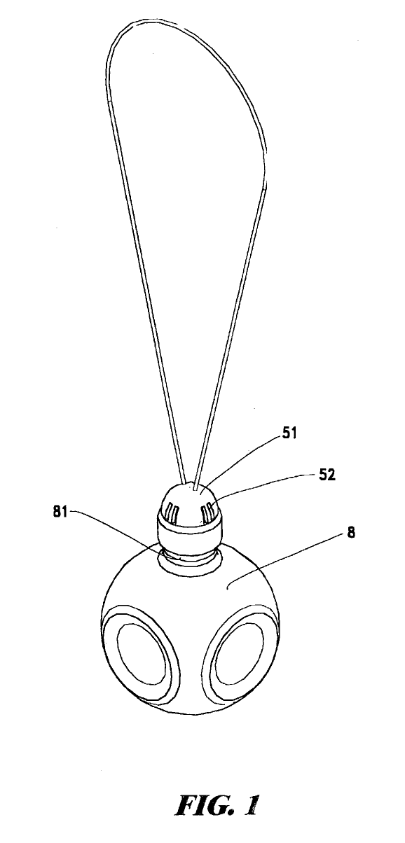

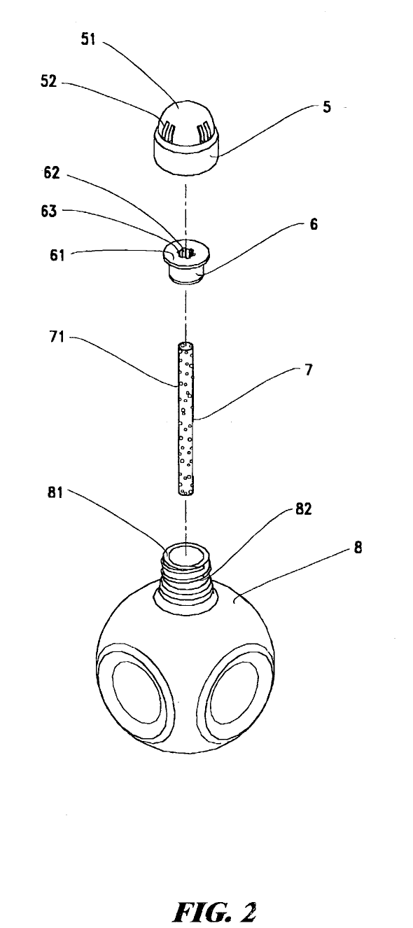

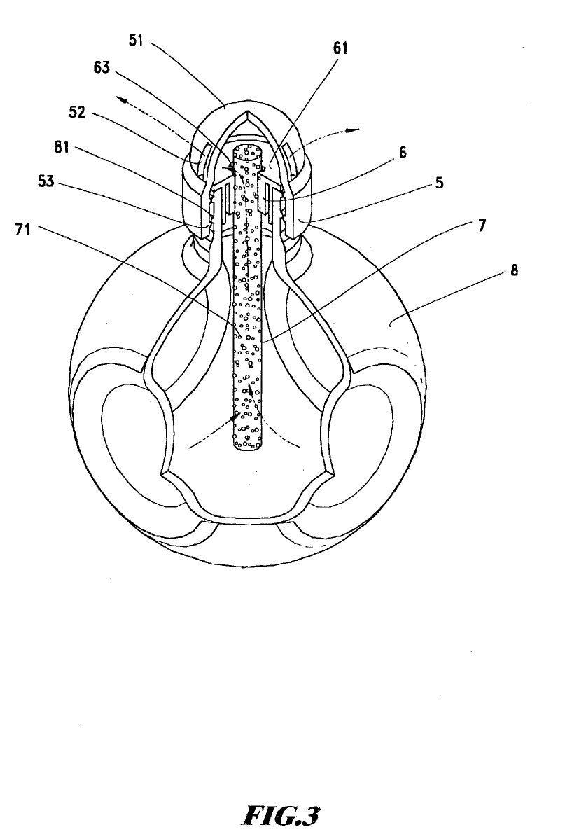

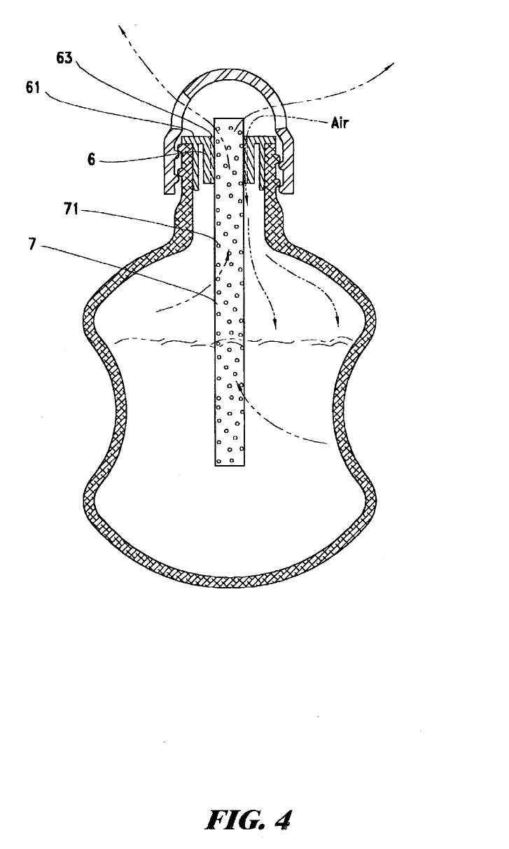

With reference to the drawings and in particular to FIGS. 1 through 4. The portable diffusing perfume bottle comprises a cap, a plug, a core pillar, a bottle for the assembly, mainly characterized in that the cap 5 covers the top circular curved surface 51 and evenly cut out a plurality of airflow openings 52 which are mutually connected inside the serrated crevice in the ringed shaped plug, and the cap has built-in female threads 53, and the bottleneck has male threads for coupling the cap by rotating the cap onto the bottleneck. The plug 6 is a round cork, and the plug plate 61 is in a ringed shape, each of the inwardly depressed teeth having a vertical ventilation passage for the core pillar to pass through and being soaked into the scented liquid such that the inner teeth of the ringed shape engage the external axis of the core pillar and it cannot easily rotate and slide out, or displace up and down. The core pillar 7 is a straight rod made of cotton material, and tiny grain-surface 71 is disposed on the axial ring of the rod to facilitate the effect of mutual engagement within the inner teeth in a ring at the central opening of the penetrating plug. It assures the axis of the core pillar extends into the scented liquid for the diffusing effect. There is space for accommodating the scent liquid in the bottle and the circular base 8, and male threads 82 are disposed at the tip of the circular base bottleneck for coupling the cap by rotation. By means of the depressed crevice in around the inner teeth in the central ring of the plug, the airflow passages are mutually connected, so that the perfume of the scented liquid can be diffused to the outside air through the opening on the cap. Such arrangement allows the core pillar to absorb the scented liquid and release the perfume to the outside air via the inside and outside circulation effect, and it also lets perfume of the scented liquid mutually vaporize and attain the practical function and performance.

Therefore, in summation of the above description, the invention provides an assembled structure that may go with the chain, bracelet, or other ornaments for added-on value by attaching the cap and hanging the perfume bottle onto those ornaments. In addition, the perfume bottle of the invention also has the function of diffusing the perfume from the portable perfume bottle. Unlike traditional perfume bottle, it has a limitation that requires the user to rub the perfume onto human skin. It may irritate our skin, or cause our body allergic or uncomfortable. The invention constitutes a special, convenient, portable bottle that can be carried pm the human body and add the value of artistic feeling by wearing it together with the ornament on human body

While the invention has been described by way of example and in terms of a preferred embodiment, it is to be understood that the invention is not limited thereto. To the contrary, it is intended to cover various modifications and similar arrangements and procedures, and the scope of the appended claims therefore should be accorded the broadest interpretation so as to encompass all such modifications and similar arrangements and procedures.

I claim:

1. A type of portable diffusing perfume bottle, comprising a cap, a plug, a core pillar, and a bottle, mainly characterized in that:
the cap, having a circular curve surface on the cap surface that evenly cuts a plurality of mutually connected airflow openings around a serrated crevice in a ring shape of the plug;
the plug, having a central opening disposed on a plate of the bottle cork, having an evenly partitioned inwardly depressed serrated crevice and forming a ringed shape surface, each of the inwardly depressed inner teeth has a vertical surface that forms an air passage;
the core pillar, being a straight rod with cotton material axial ringed surface having a tiny grain-surface for facilitating the penetration of the inner teeth in the ring shape of the central opening, and generating an engagement to each other in order to assure the core pillar extends into the scented liquid for absorption and vaporization effect;
the bottle, having a circular neck at it end for coupling the cap;
by means of the inwardly depressed crevice being disposed in the ring shape of the plug, it forms a plurality of mutually connected airflow passage to facilitate the perfume of the scented liquid in the bottle to pass to the air through the airflow openings of the cap, and allow the core pillar to absorb the scented liquid and diffuse to the outside air that generates an in-and-out circulation effect, such that the perfume of the scented liquid in the perfume bottle can have the effect of being vaporized interactively.
